(19) United States
(12) Patent Application Publication
(10) Pub. No.: US 2026/0053546 A1
(43) Pub. Date: Feb. 26, 2026

(54) SYSTEM TO REDUCE CURRENT LOSS TO A SURROUNDING CONDUCTIVE MEDIUM FOR PURPOSES OF PUNCTURING TISSUE

(71) Applicant: Boston Scientific Scimed, Inc., Maple Grove, MN (US)

(72) Inventors: Christian BALKOVEC, Cambridge (CA); Gareth DAVIES, Toronto (CA)

(21) Appl. No.: 19/305,429

(22) Filed: Aug. 20, 2025

Related U.S. Application Data
(60) Provisional application No. 63/685,656, filed on Aug. 21, 2024.

(51) Int. Cl. A61B 18/14; A61B 18/00

(52) U.S. Cl. CPC A61B 18/1477; A61B 18/1492; A61B 2018/00083; A61B 2018/00351; A61B 2018/00625; A61B 2018/147; A61B 2018/1475

(57) ABSTRACT

An electrosurgical system for puncturing tissue includes an electrosurgical generator configured to generate radiofrequency (RF) energy and a crossing device connected to the electrosurgical generator. The crossing device includes an electrode positioned at a distal tip of the crossing device, and an electrically insulating cover that is elastically compliant, the cover extending distally beyond a distal end of the electrode when the electrode is in a stowed configuration, and the electrode extending distally beyond a distal end of the cover when the electrode is in a deployed configuration.

### CROSS REFERENCE TO RELATED APPLICATIONS

This application claims priority to U.S. Provisional Application No. 63/685,656 entitled “SYSTEM TO REDUCE CURRENT LOSS TO A SURROUNDING CONDUCTIVE MEDIUM FOR PURPOSES OF PUNCTURING TISSUE,” filed Aug. 21, 2024, which is hereby incorporated by reference in its entirety.

### TECHNICAL FIELD

The present disclosure relates to medical systems and methods for vaporizing tissue in a patient. More specifically, the present disclosure relates to medical systems and methods for puncturing bodily tissues such as the atrial septum with an electrode by dielectric breakdown.

### BACKGROUND

Minimally invasive surgical techniques can employ catheters to provide access into a patient's body. Surgical instruments can be routed through the catheters such that the instruments can be positioned where they are needed for a given procedure. For example, in a cardiac procedure involving the left atrium of the heart, it may be advantageous to route a catheter into the right atrium and perform a transseptal puncture procedure to enter the left atrium. Such a transseptal puncture procedure can be performed using one or more electrodes that are passed through a catheter and energized in the radiofrequency (RF) range. While this procedure can vaporize the targeted septal tissue, it can also vaporize liquids (e.g., blood) that are in contact with the electrode(s).

### SUMMARY

In Example 1, An electrosurgical system for puncturing tissue, the electrosurgical system comprising: an electrosurgical generator configured to generate radiofrequency (RF) energy; and a crossing device connected to the electrosurgical generator, the crossing device comprising: an electrode positioned at a distal tip of the crossing device; an electrically insulating cover that is elastically compliant, the cover extending distally beyond a distal end of the electrode when the electrode is in a stowed configuration, and the electrode extending distally beyond a distal end of the cover when the electrode is in a deployed configuration.

In Example 2, the electrosurgical system of Example 1, wherein the cover comprises a longitudinal tube that extends along a side of the electrode when the electrode is in a stowed position.

In Example 3, the electrosurgical system of Example 2, wherein: the cover comprises a plurality of leaflets connected to the longitudinal tube and extending transversely over the distal end of the electrode when the electrode is in the stowed configuration; and the plurality of leaflets is spread apart when the electrode is in the deployed configuration.

In Example 4, the electrosurgical system of Examples 2 or 3, wherein the leaflets meet to seal the electrode when the electrode is in the stowed configuration.

In Example 5, the electrosurgical system of any of Examples 2-4, wherein the cover extends at least about one-quarter of a diameter of the electrode beyond the distal end of the electrode when the electrode is in the stowed configuration.

In Example 6, the electrosurgical system of any of Examples 2-5, wherein the leaflets are equally sized.

In Example 7, the electrosurgical system of Examples 1 or 2, wherein the cover is corrugated when the electrode is in the deployed configuration.

In Example 8, the electrosurgical system of Example 7 and Examples 1 or 2, wherein the cover includes a fold line that is a weakened section of the cover.

In Example 9, the electrosurgical system of Examples 1 or 2, wherein: the distal end of the cover includes a plurality of slits defining a plurality of petals; and the distal end of the cover is splayed when the electrode is in the deployed configuration.

In Example 10, the electrosurgical system of Example 9 and Examples 1 or 2, wherein the petals are equally sized.

In Example 11, the electrosurgical system of Example 9 and any of Examples 1, 2, or 10, wherein the plurality of slits extends at least about one-half of a diameter of the electrode proximally beyond the distal end of the electrode when the electrode is in the stowed configuration.

In Example 12, the electrosurgical system of any of Examples 1-11, wherein the cover is slidably positioned on the electrode.

In Example 13, the electrosurgical system of any of Examples 1-12, wherein a proximal portion of the cover is more rigid than a distal portion of the cover.

In Example 14, the electrosurgical system of any of Examples 1-13, wherein the crossing device is a wire.

In Example 15, the electrosurgical system of any of Examples 1-13, wherein the crossing device is a needle.

In Example 16, an electrosurgical system for puncturing tissue, the electrosurgical system comprising: an electrosurgical generator configured to generate radiofrequency (RF) energy; and a crossing device connected to the electrosurgical generator, the crossing device comprising: an electrode positioned at a distal tip of the crossing device; an electrically insulating cover that is elastically compliant, the cover extending distally beyond a distal end of the electrode when the electrode is in a stowed configuration, and the electrode extending distally beyond a distal end of the cover when the electrode is in a deployed configuration.

In Example 17, the electrosurgical system of Example 16, wherein the cover comprises a longitudinal tube that extends along a side of the electrode when the electrode is in a stowed position.

In Example 18, the electrosurgical system of Example 17, wherein: the cover comprises a plurality of leaflets connected to the longitudinal tube and extending transversely over the distal end of the electrode when the electrode is in the stowed configuration; and the plurality of leaflets is spread apart when the electrode is in the deployed configuration.

In Example 19, the electrosurgical system of Example 17, wherein the cover extends at least about one-quarter of a diameter of the electrode beyond the distal end of the electrode when the electrode is in the stowed configuration.

In Example 20, the electrosurgical system of Example 17, wherein the cover is corrugated when the electrode is in the deployed configuration.

In Example 21, the electrosurgical system of Example 17, wherein: the distal end of the cover includes a plurality of slits defining a plurality of petals; and the distal end of the cover is splayed when the electrode is in the deployed configuration.

In Example 22, the electrosurgical system of Example 16, wherein the cover is slidably positioned on the electrode.

In Example 23, the electrosurgical system of Example 16, wherein the crossing device is a wire.

In Example 24, the electrosurgical system of Example 16, wherein the crossing device is a needle.

In Example 25, a crossing device for an electrosurgical system for puncturing tissue, the crossing device comprising: a conductor configured to be connected to an electrosurgical generator that is configured to generate radiofrequency (RF) energy; an electrode electrically connected to the conductor and positioned at a distal end of the crossing device; an electrically insulating cover that is elastically compliant and extends over the conductor and the electrode, the cover extending distally beyond a distal end of the electrode when the electrode is in a stowed configuration, and the electrode extending distally beyond a distal end of the cover when the electrode is in a deployed configuration.

In Example 26, the crossing device of Example 25, wherein the cover comprises a longitudinal tube that extends along a side of the electrode when the electrode is in a stowed position.

In Example 27, the crossing device of Example 26, wherein: the cover comprises a plurality of leaflets connected to the longitudinal tube and extending transversely over the distal end of the electrode when the electrode is in the stowed configuration; and the plurality of leaflets is spread apart when the electrode is in the deployed configuration.

In Example 28, the crossing device of Example 25, wherein the cover extends at least about one-quarter of a diameter of the electrode beyond the distal end of the electrode when the electrode is in the stowed configuration.

In Example 29, the electrosurgical system of Example 25, wherein the crossing device is a needle.

In Example 30, a method of operating an electrosurgical system for puncturing tissue, the method comprising: deploying an electrode from an elastically compliant cover to expose an electrode; energizing the electrode an electrosurgical generator; and vaporizing tissue with the energized electrode.

In Example 31, the method of Example 30, wherein the electrode is positioned near to the tissue but not in contact with the tissue prior to energizing the electrode.

In Example 32, the method of Example 30, wherein the electrode is positioned in contact with the tissue prior to energizing the electrode.

In Example 33, the method of Example 30, wherein deploying the electrode comprises advancing the electrode and spreading apart a plurality of leaflets of the cover.

In Example 34, the method of Example 30, wherein deploying the electrode comprises retracting the cover.

In Example 35, the method of Example 30, wherein deploying the electrode comprises splaying a plurality of petals of the cover.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a schematic diagram illustrating an example electrosurgical system for treating a patient, such as a heart or the vasculature of a patient, including an electrosurgical generator and a transseptal crossing system, consistent with various aspects of the present disclosure.

FIG. 2 is a cutaway view of a heart including an atrial septum and the transseptal crossing system, consistent with various aspects of the present disclosure.

FIG. 3 is a side view of the distal end of the transseptal crossing system, consistent with various aspects of the present disclosure.

FIGS. 4A-4C are views of the distal tip of the transseptal crossing system. More specifically, FIG. 4A is a cross-sectional view as indicated by line 4-4 in FIG. 4B of the distal tip in a stowed configuration, consistent with various aspects of the present disclosure. FIG. 4B is a front view of a distal tip of the transseptal crossing system, consistent with various aspects of the present disclosure. FIG. 4C is a cross-sectional view as indicated by line 4-4 in FIG. 4B of the distal tip in a deployed configuration, consistent with various aspects of the present disclosure.

FIG. 5 is a flowchart of a method of crossing the atrial septum, consistent with various aspects of the present disclosure.

FIGS. 6A and 6B are a series of schematic diagrams of operations of crossing the atrial septum according to the method of FIG. 5, consistent with various aspects of the present disclosure.

FIG. 7 is a flowchart of a method of operating the electrosurgical system to puncture tissue, consistent with various aspects of the present disclosure.

FIG. 8A-8C are a series of schematic diagrams of operations of puncturing tissue according to the method of FIG. 7, consistent with various aspects of the present disclosure.

FIG. 9 is a cross-sectional view of an alternative distal tip of the transseptal crossing system in a stowed configuration, consistent with various aspects of the present disclosure.

FIG. 10 is a flowchart of an alternative method of crossing the atrial septum, consistent with various aspects of the present disclosure.

FIG. 11A-11D are a series of schematic diagrams of operations of puncturing tissue according to the alternative method of FIG. 10, consistent with various aspects of the present disclosure.

FIG. 12 is a side view of an alternative distal tip of the transseptal crossing system in a stowed configuration, consistent with various aspects of the present disclosure.

FIG. 13 is a flowchart of an alternative method of operating an alternative electrosurgical system to puncture tissue, consistent with various aspects of the present disclosure.

FIGS. 14A and 14B are a series of schematic diagrams of operations of puncturing tissue according to the alternative method of FIG. 10, consistent with various aspects of the present disclosure.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) the features in a given example used across all examples. Thus, no one figure should be interpreted as having any dependency or requirement related to any single component or combination of components illustrated therein. Additionally, various components depicted in a given figure may be, in examples, integrated with various ones of the other components depicted therein (and/or components not illustrated), all of which are considered to be within the ambit of the present disclosure.

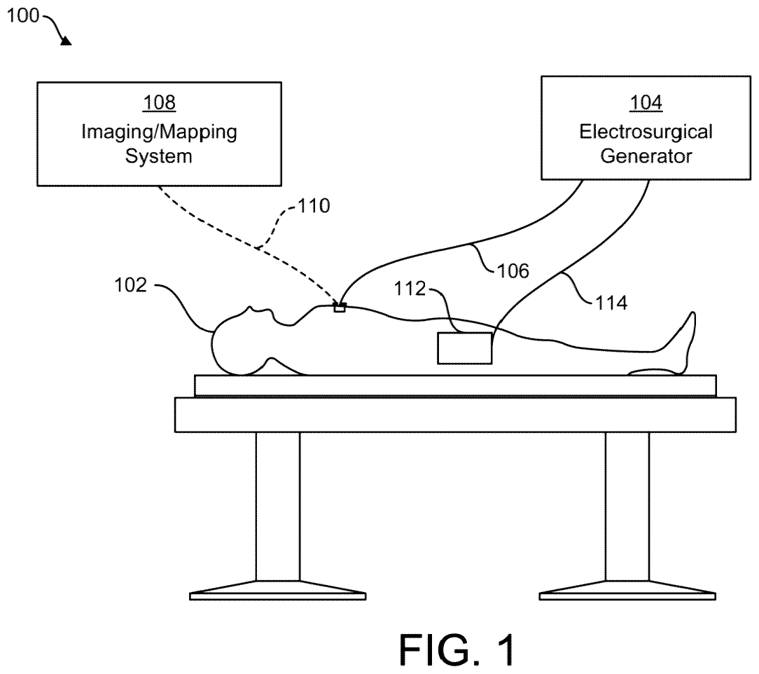

FIG. 1 shows an electrosurgical system 100 for treating a patient 102. In the illustrated embodiment, the system 100 includes an electrosurgical generator 104 with a transseptal crossing system 106 and an imaging/mapping system 108 for tracking the crossing system 106 in the patient 102. The imaging/mapping system 108 can use an external fluoroscopy system (not shown) and/or a mapping catheter 110 (shown in phantom) (such as, for example, the OPAL HDx™ mapping system from the Boston Scientific Corporation).

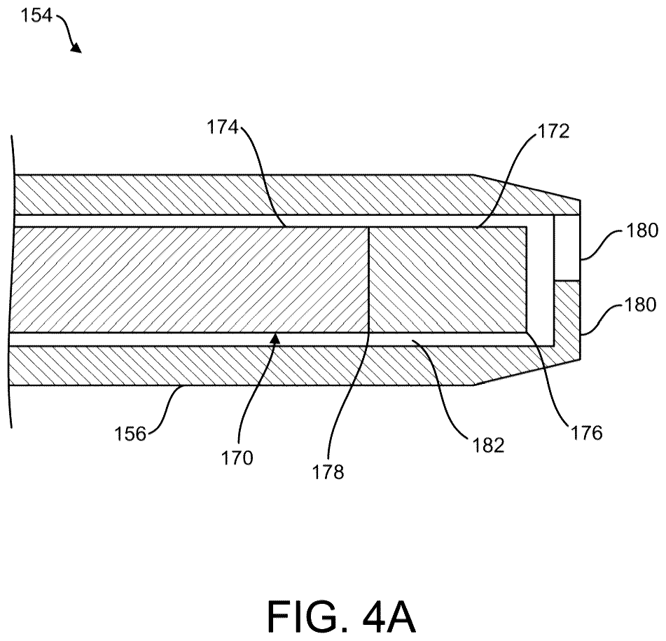

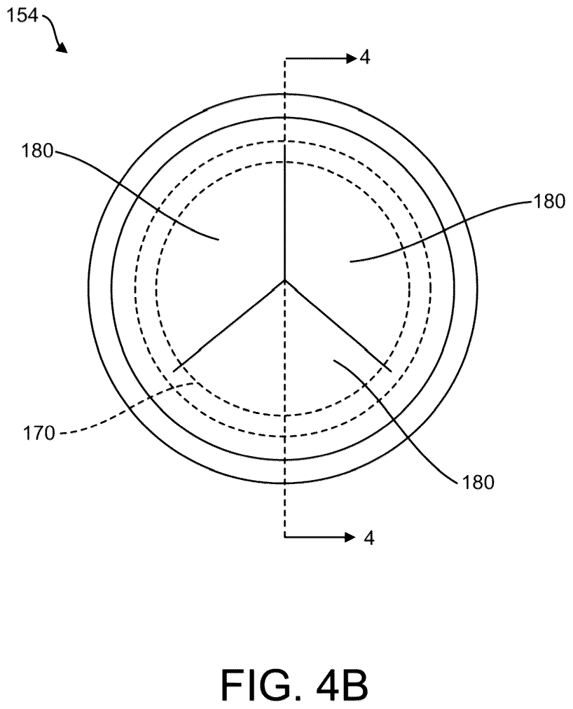

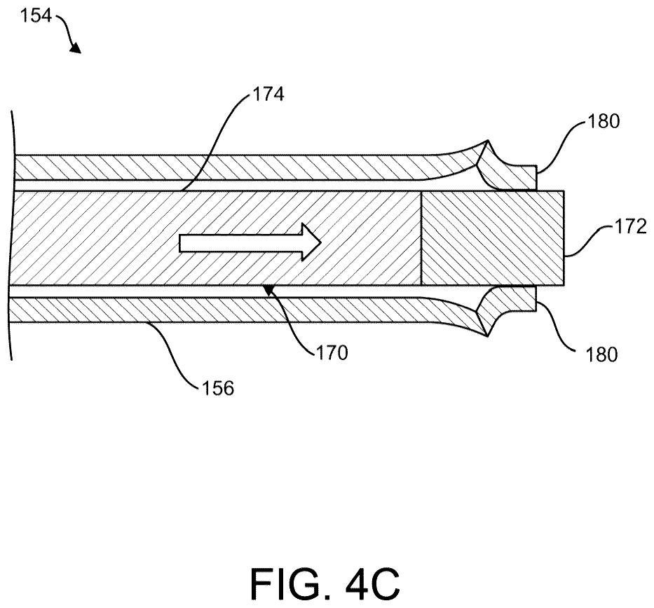

In the illustrated embodiment, the electrosurgical generator 104 is configured to provide energy, such as radiofrequency (RF) electrical energy, to the crossing system 106. Typically, the conductive crossing system 106 is electrically insulated with the exception of a small distal portion, formed as a vaporizing electrode (shown in FIG. 4A), that is intended to deliver the RF energy to the target tissue. To achieve tissue vaporization, the target tissue is rapidly heated. If heating is too slow, the tissue is desiccated rather than vaporized. Rapid heating is achieved through high current density at the electrode-tissue interface, meaning the electrode is either relatively small and/or the energy delivered is relatively high.

The delivery of energy to the target tissue can also heat the crossing system 106 itself. Thus, the electrical insulation around the crossing system 106 (especially near the electrode) should be able to withstand such heat without breaking down. While typically the insulation has been made from per- and polyfluoroalkyl substances (PFAS) (e.g., polytetrafluoroethylene (PTFE)), other materials may have the advantage of being less environmentally problematic. Such alternative materials, however, may lack the heat performance of the traditional materials, so the crossing system 106 should be designed to reduce the heat generation within itself while still providing adequate heat generation in the target tissue.

In the illustrated embodiment of FIG. 1, the crossing system 106 is monopolar and includes a single vaporizing electrode (i.e., an active electrode), so the system 100 also includes a patch electrode 112 (i.e., an indifferent or dispersive electrode). The patch electrode 112 has a large surface area to lower current density, so the patch electrode 112 is typically located on the back, buttocks, or upper leg of the patient 102, although there may be other suitable locations as well. The patch electrode 112 returns the RF electrical energy to the generator 104 through the lead 114. In some embodiments, the RF energy for a monopolar puncture function is provided by the electrosurgical generator 104 at a selected voltage and a continuous current (100% on, or 100% duty cycle). For example, if a power setting of 50 watts (W) is used for puncturing (which can mean that the instantaneous power is higher than 50 W), the voltage can range from approximately 164 volts (V) to 400 V root mean square (RMS).

In addition, the electrosurgical generator 104 can include a plurality of functions and provide programmed and custom settings via an interface (not shown). For example, the electrosurgical generator 104 provides RF energy to the crossing system 106 as an alternating current having a frequency in the range of 100 kilohertz (kHz) to 10 megahertz (MHz). Such puncturing RF energy can be applied in the form of a continuous waveform signal or in bursts of a waveform signal. In the latter case, the individual bursts of the waveform signal can have a duration of about 300 milliseconds (ms) with a rest interval between pulses of about 700 ms, although other durations of bursts and intervals can be used. In some embodiments, the waveform signals themselves can be sinusoidal or square waves that are bi-phasic. Furthermore, the electrosurgical generator 104 can be couplable to other electrosurgical tools and/or the electrosurgical generator 104 can receive signals (e.g., from the crossing system 106) to monitor the patient 102.

The components and configuration of electrosurgical system 100 allow for target tissue to be vaporized. In some embodiments, the tissue vaporization allows the crossing system 106 to puncture through the atrial septum for treatment of the left side of the heart of the patient 102. While examples of the devices, systems, and methods of the present disclosure are presented in the context of a transeptal puncture, a person having ordinary skill in the art will recognize other applicable contexts. For example, the electrosurgical systems of the present disclosure can be employed to puncture a pericardium layer of a patient for epicardial access and/or to remove accumulation of atheromatous material on the inner walls of vascular lumens.

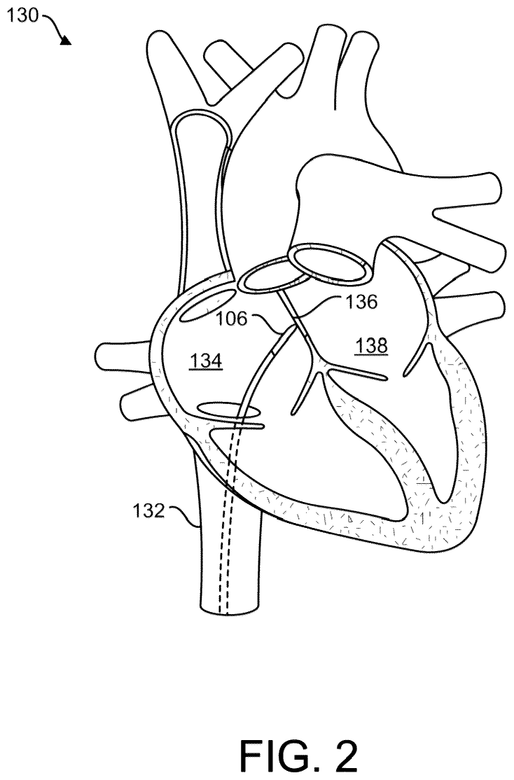

FIG. 2 shows a heart 130 of the patient 102 (shown in FIG. 1) with selected portions cut away. In the illustrated embodiment, the crossing system 106 extends through the inferior vena cava 132 from a surgical entry site (not shown) that is distal to the heart 130. The distal end of the crossing system 106 is positioned in the right atrium 134 and is in contact with the atrial septum 136. In the illustrated embodiment, the tip of the crossing system 106 is positioned at the fossa ovalis since this region of the atrial septum 136 is relatively safe and easy to puncture. Once the atrial septum 136 is crossed, the physician (not shown) will have access to the left atrium 138 (e.g., for treatment thereof).

In some use cases, such as a septal crossing, the electrode will be surrounded by a conductive liquid medium, such as blood, that is near the target tissue. When the crossing system 106 is operating in such an environment, any surface of the electrode that is in contact with the conductive liquid (instead of with the target tissue) provides a shunt path for the electrical current. These alternative electrical pathways do not help vaporize the target tissue, so they decrease the efficiency of the tissue vaporization process. Furthermore, these pathways can cause the blood to locally coagulate and form thrombotic material that can lead to embolization and subsequent deleterious health effects for the patient 102 (shown in FIG. 1). Therefore, components and methods to prevent such occurrences are discussed in the present disclosure.

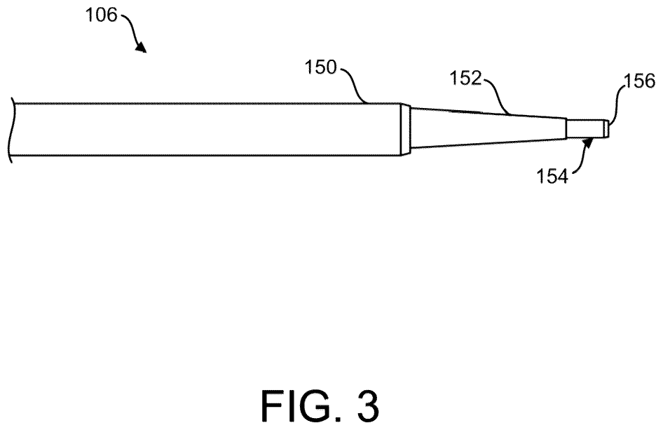

FIG. 3 shows a distal end of the crossing system 106. In the illustrated embodiment, the crossing system 106 includes a sheath 150, a dilator 152, and a crossing device 154. The crossing device 154 is an elongated tissue vaporization device that can have the form of, for example, a wire, a needle, forceps, scalpels, or other devices that puncture and/or cut tissue. In some embodiments, a wire is a solid, stiff but elastically deformable member with a generally straight and/or helical configuration. In some embodiments, a needle is a hollow, flexible member with a generally straight configuration through which fluid can be pumped. The fluid can exit near an electrode that is positioned at the distal end of the needle, and the electrode can be connected to the electrosurgical generator 104 with a conductor since the flexible member can be made from an electrically insulating material. In some embodiments, forceps are a dual-levered instrument capable of grasping and/or holding tissue or other objects between their distal ends. In some embodiments, a scalpel is a bladed instrument with a sharpened edge capable of cutting tissue or other objects.

In some embodiments, the crossing system 106 has an overall length between about 55 centimeters (cm) and about 300 cm. The sheath 150 is an elongate member with a central lumen (not shown), in which the dilator 152 and the crossing device 154 are slidably positioned. The central lumen diameter is similar to the outer diameter of the majority of the dilator 152 (except for the distal tip), and the sheath 150 is tapered at the distal end to make the transition between the sheath 150 and the dilator 152 smoother. In addition, the sheath 150 can be a steerable sheath and/or have a fixed or adjustable curve at the distal end for positioning of the dilator 152 and the crossing device 154. In some embodiments, the sheath 150 and the dilator 152 are generally similar to those of the VersaCross™ Access Solution from Boston Scientific.

In the illustrated embodiment, the dilator 152 is an elongate member with a central lumen (not shown), in which the crossing device 154 is slidably positioned. The central lumen diameter is similar to the outer diameter of the crossing device 154, and the dilator 152 is tapered at the distal end to make the transition between the dilator 152 and the crossing device 154 smoother. In addition, the dilator 152 can have a fixed or adjustable curve at the distal end for positioning of the crossing device 154 against the atrial septum 136 (shown in FIG. 2).

In the illustrated embodiment, the crossing device 154 is an elongate member with an electrically insulating, compliant cover 156 over a central member (shown in FIG. 4A). The cover 156 comprises, for example, parylene, polyimide, polyethylene terephthalate (PET), polyurethane, silicone ((R 2 SiO) x), high density polyethylene (HDPE), low density polyethylene (HDPE), PTFE heat shrink, or combinations thereof. In some embodiments, a diameter of the crossing device 154 is between about 0.50 millimeters (mm) and about 1.0 mm. Furthermore, in other embodiments, the cover is an integral portion of the dilator. In such embodiments, the crossing device would not include its own separate cover. Furthermore, in such embodiments, the dilator could be comprised of different materials in different regions (e.g., the distal cover region can be made from silicone whereas the remainder of the dilator can be made from HDPE).

FIG. 4A shows the crossing device 154 with the cover 156 and the central member 170 slidably positioned within the cover 156 since the distal tip is in a stowed configuration. In the illustrated embodiment, the central member 170 comprises an electrode 172 at the distal end and a conductor 174 that is connected to the electrode 172. The electrode 172 is electrically connected to the electrosurgical generator 104 (shown in FIG. 1) via the conductor 174. Thereby, the central member 170 comprises, for example, stainless steel, nitinol, platinum, gold, iridium, or combinations thereof. In some embodiments, the conductor 174 includes its own electrical insulation, but in other embodiments, the conductor 174 is solely electrically insulated by the cover 156. In addition, the electrode 172 has a cylindrical shape with a flat distal end having a distal circular edge 176 and a proximal circular edge 178 where the proximal end of the electrode 172 and the distal end of the conductor 174 are coterminous. Thus, the electrode 172 is atraumatic (i.e., not sharp) and has distinct circular edges 176, 178 at the distal and proximal ends, respectively. In other embodiments, however, the crossing device has a distal domed shape.

In the illustrated embodiment, the cover 156 includes a plurality of leaflets 180 at the distal end. The leaflets 180 extend radially inward from the circumference of the cover 156. When the electrode 172 is in the stowed configuration (as it is shown in FIG. 4A), the cover 156 is the most distal portion of the crossing device 154.

In the illustrated embodiment, there is a gap 182 between the central member 170 and the cover 156. The size of the gap 182 can be moderate (as shown in FIG. 4A), or, in other embodiments, the gap 182 can be larger, smaller, or essentially nonexistent.

FIG. 4B shows that the cover 156 includes three equally sized leaflets 180, although in other embodiments, there are greater or fewer numbers of leaflets and/or the sizes of the leaflets can vary. In the illustrated embodiment, the leaflets 180 meet in the center to seal the inside of the cover 156. The sealing prevents incursion of the conductive liquid medium that surrounds the crossing device 154 when the crossing device 154 is in the heart 130 (shown in FIG. 2). Even if the leaflets 180 do not completely seal the conductive liquid medium from the crossing device 154, the leaflets 180 electrically insulate the distal end of the crossing device 154 from the bulk of the conductive liquid medium.

FIG. 4C shows the crossing device 154 in a deployed configuration. In the illustrated embodiment, the physician has exerted a distally-oriented force on the central member 170 causing the electrode 172 to advance. Upon advancing, the leaflets 180 are pushed aside since the leaflets 180 are made from compliant material that is elastically deformable to accommodate the electrode 172 moving out of the distal end of the cover 156. Thus, the electrode 172 is the most distal portion of the crossing device 154 when the crossing device 154 is in the deployed configuration. Furthermore, the leaflets 180 cover portions of the sides of the electrode 172 when the crossing device 154 is in the deployed configuration, and the leaflets 180 can return to their original positions when the crossing device 154 is returned to the stowed configuration. In some embodiments, the entire cover 156 is made from a compliant material, but in other embodiments, only the leaflets 180 (or the distal end portion) are compliant and the remainder of the cover 156 is rigid.

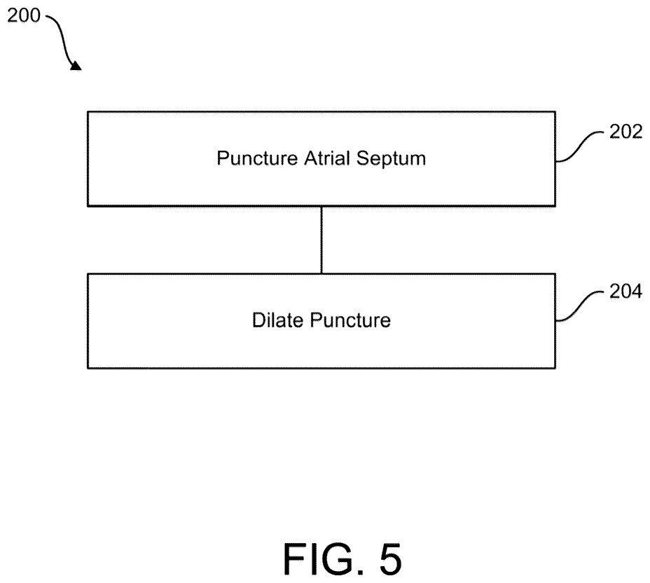

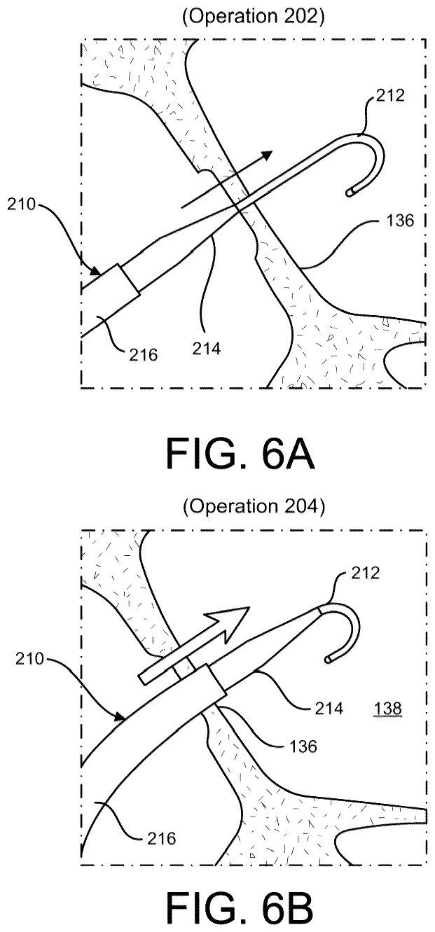

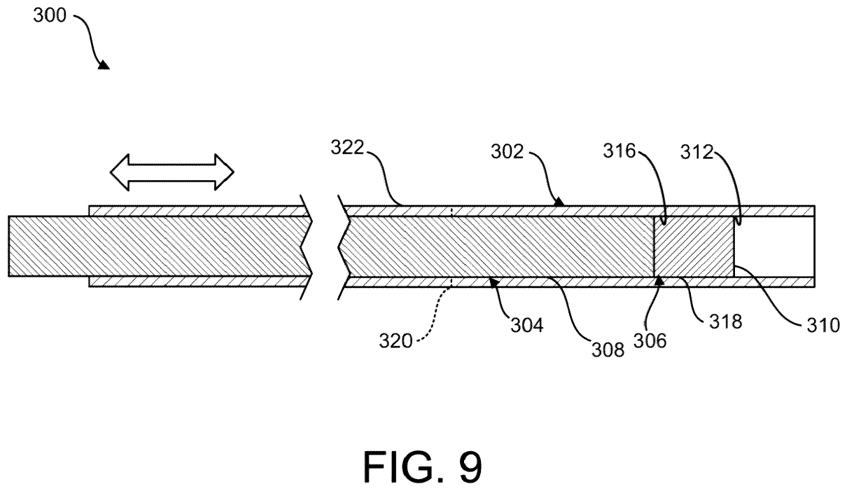

FIG. 5 shows a method 200 of crossing the atrial septum 136 using the crossing system 210 which can be the same as or similar to the crossing system 106 (shown in FIG. 2). FIGS. 6A and 6B show the operations of crossing the atrial septum 136. The distal tip of the crossing system 210 can be the same as or similar to the crossing device 154 (shown in FIG. 4C) or a different crossing device can be used (e.g., the crossing device shown in FIG. 9). FIGS. 5 and 6A and 6B will now be discussed in conjunction with one another, and each operation of the method 200 is illustrated by a corresponding one of FIGS. 6A and 6B.

In the illustrated embodiment, the method 200 begins with a crossing system 210 being positioned near the atrial septum 136. At operation 202, as shown in FIG. 6A, the crossing device 212 is energized by the electrosurgical generator 104, and the physician applies distally-oriented force to the crossing device 212 (as indicated by the arrow). Thus, the crossing device 212 is forced distally by the physician punctures through the atrial septum 136, and the crossing device 212 is deenergized. As shown in FIG. 6A, the crossing device 212 has a J-shape that is elastically deformed to be straight when the crossing device 212 is in the dilator 214, so the crossing device 212 returns to the J-shape when the crossing device 212 exits the dilator 214.

At operation 204, as shown in FIG. 6B, the physician applies distally-oriented force to the dilator 214 and to the sheath 216 (as indicated by the arrow). Thus, the dilator 214 and the sheath 216 follow the crossing device 212 through the atrial septum 136. As the dilator 214 follows the crossing device 212, the puncture in the atrial septum 136 is gently stretched by the dilator 214 so that the sheath 216 can enter the left atrium 138. Once the sheath 216 is positioned in the left atrium 138, one or more of the crossing device 212, the dilator 214, and the sheath 216 can be withdrawn and replaced with another component, such as, for example, a left atrial appendage closure (LAAC) implant device (not shown).

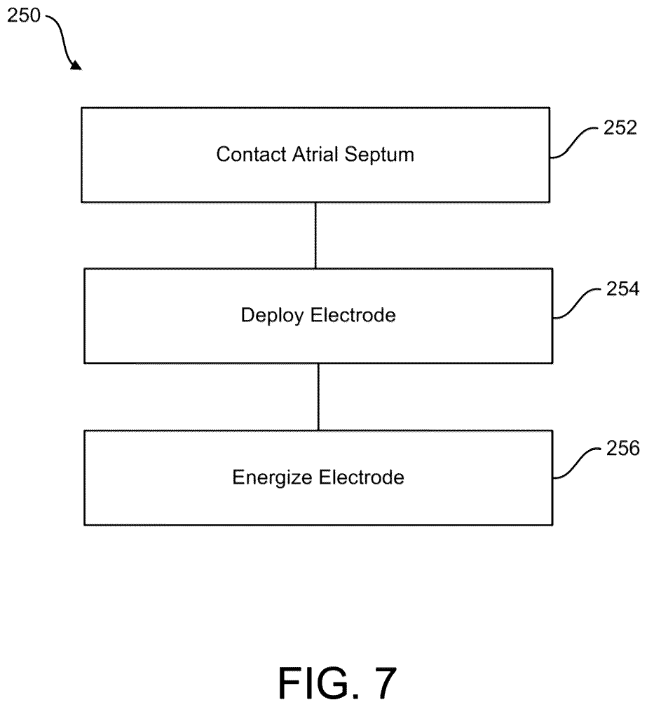

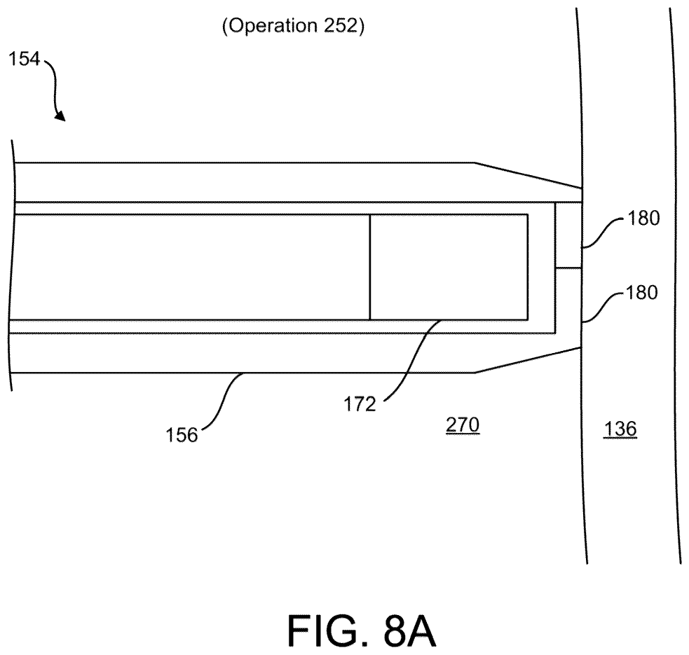

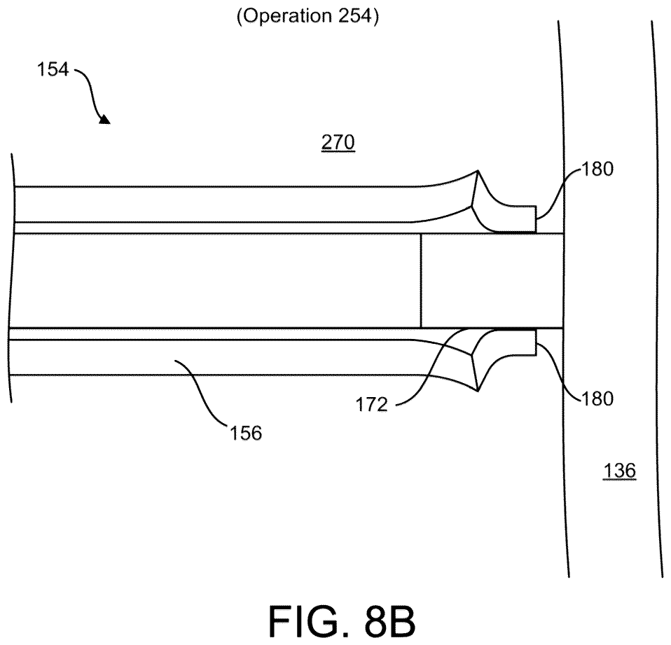

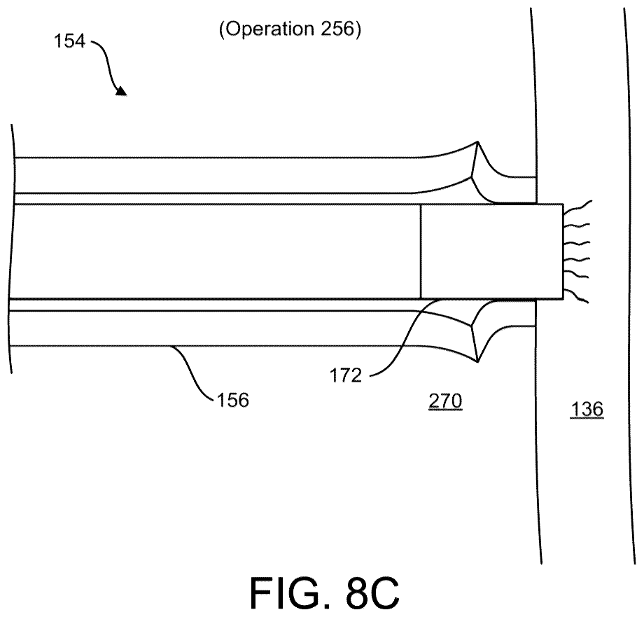

FIG. 7 shows a method 250 of puncturing the atrial septum 136. FIGS. 8A-8F show the operations of puncturing the atrial septum 136 using the crossing device 154. FIGS. 7 and 8A-8C will now be discussed in conjunction with one another, and each operation of the method 250 is illustrated by a corresponding one of FIGS. 8A-8C.

In the illustrated embodiment, the method 250 begins with operation 252 wherein the crossing device 154 is positioned in direct contact with the atrial septum 136. As shown in FIG. 8A, the electrode 172 is in the stowed configuration, so the leaflets 180 are positioned between the electrode 172 and the atrial septum 136, and the cover 156 is positioned between the electrode 172 and the blood 270. Furthermore, in other embodiments where the cover is part of the dilator, then the first operation would be positioning the dilator in direct contact with the atrial septum 136.

At operation 254, as shown in FIG. 8B, the crossing device 154 is advanced by the physician. The crossing device 154 pushes the leaflets 180 aside and extends beyond the distal end of the cover 156, deploying the electrode 172. Thus, the distal end of the electrode 172 contacts the atrial septum 136, and the distally-oriented force from the physician on the crossing device 154 slightly tents the atrial septum 136.

At operation 256, as shown in FIG. 8C, the physician has activated the electrosurgical generator 104 (shown in FIG. 1) which energizes the electrode 172. A substantial portion of the RF energy being transmitted to the electrode 172 flows into the cells of the atrial septum 136 that are closest to the electrode 172, vaporizing the cells. At first, some of the RF energy is also transmitted into the blood 270 due to the gap between the cover 156 and the atrial septum 136. As the cells of the atrial septum 136 are vaporized at the distal end of the electrode 172, the crossing device 154 penetrates into the atrial septum 136.

Penetration into the atrial septum 136 closes the gap between the cover 156 and the atrial septum 136, so the electrode 172 is no longer in direct contact with the blood 270. Thereby, the electrode 172 is electrically insulated from the blood 270. Virtually all of the RF energy being transmitted to the electrode 172 flows into the cells of the atrial septum 136 which increases the efficiency and ease of the crossing. In particular, the crossing device 154 penetrates through the atrial septum 136 with minimal tenting of the atrial septum 136 and with minimal jumping of the crossing device 154 upon breakthrough. The ease of the puncture means that the physician does not need to exert as much force on the crossing device 154 compared to a prior art crossing system that has inefficient or diffuse RF energy delivery. The inefficiency or diffuseness of the prior art can result from insufficient insulation of the electrode from the blood 270. Such an electrode would maintain contact with the blood 270 even as it penetrates into the atrial septum 136, so only a fraction of the RF energy provided to the electrode would be delivered to the atrial septum 136. The inefficient or diffuse delivery of the RF energy can result in the tissue cells receiving an insufficient amount of power for vaporization but enough for the tissue cells to be desiccated instead. Desiccation increases the electrical resistance of the affected tissue cells, because there are fewer polar water molecules in the cells to conduct electricity, so the result is that even more power flows into the blood 270 instead of into the atrial septum 136.

In an alternative embodiment to method 250, the order of the operations is different. For example, in some embodiments, the electrode 172 is energized in the stowed configuration, and then the electrode 172 is deployed into the atrial septum 136. In such an embodiment, there would be an additional gaseous insulation layer formed prior to contact with the atrial septum that would be destroyed during the puncturing.

FIG. 9 shows an alternative crossing device 300 with a cover 302 and a central member 304 slidably positioned within the cover 302 since the device 300 is in a stowed configuration. In the illustrated embodiment, the central member 304 comprises an electrode 306 at the distal end and a conductor 308 that is connected to the electrode 306. The electrode 306 is electrically connected to the electrosurgical generator 104 (shown in FIG. 1) via the conductor 308. Thereby, the central member 304 comprises, for example, stainless steel, nitinol, platinum, gold, iridium, or combinations thereof. In some embodiments, the conductor 308 includes its own electrical insulation, but in other embodiments, the conductor 308 is solely electrically insulated by the cover 302. In addition, the electrode 306 has a cylindrical shape with a flat distal end surface 310 having a distal circular edge 312, so the electrode 306 is atraumatic (i.e., not sharp). In other embodiments, however, the electrode has a distal domed shape.

In the illustrated embodiment, the cover 302 is made from a compliant, electrically insulative material that extends distally beyond a distal end surface 310 of the electrode 306 when the electrode 306 is in the stowed configuration (as it is shown in FIG. 9). In some embodiments, the cover 302 extends at least one-quarter of a diameter (e.g., at least about 0.1 mm to 0.3 mm) beyond the distal end surface 310. There is little to no gap between the circumferential interior 316 of the cover 302 and the circumferential exterior 318 of the electrode 306 when the electrode 306 is in the stowed configuration. Thus, the circumferential exterior 318 is electrically insulated by the cover 302 when the electrode 306 is in the stowed configuration, so the conductive fluid medium cannot contact the circumferential exterior 318. The conductive fluid medium, however, can contact the distal end surface 310, because the distal end of the cover 302 is open.

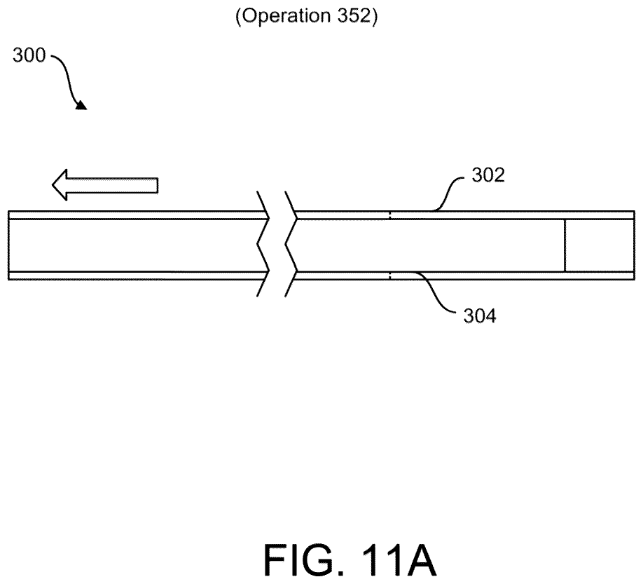

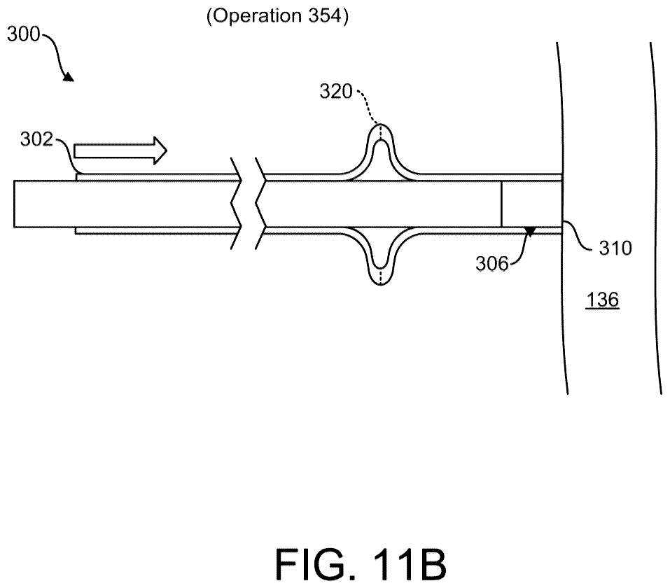

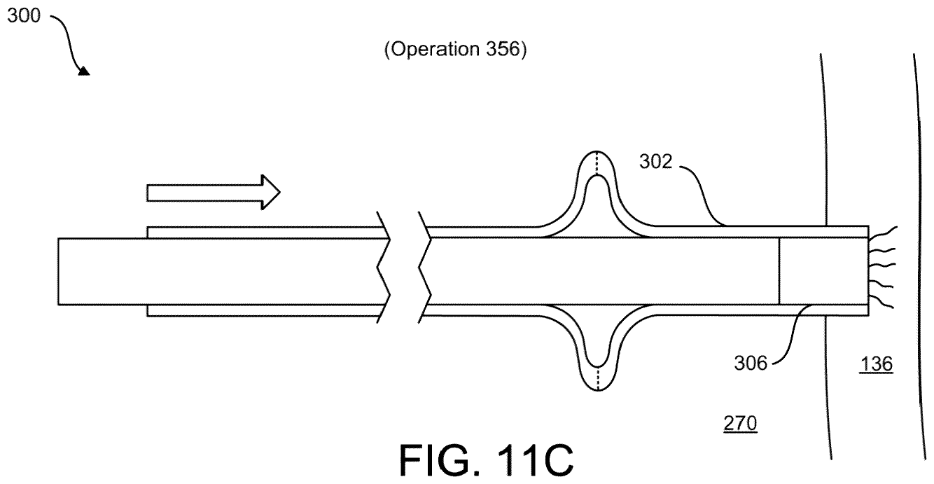

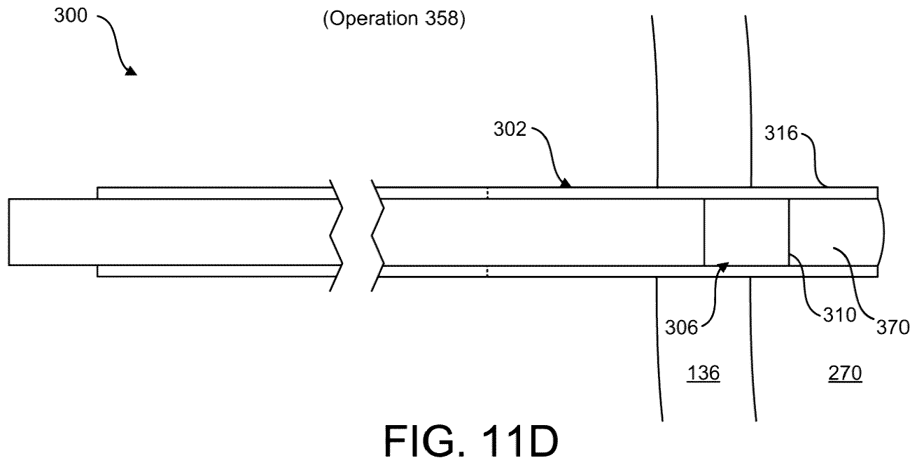

In the illustrated embodiment, the cover 302 includes a fold line 320 around the circumference of the cover 302 at a selected longitudinal location near the distal end of the cover 302. In some embodiments, the fold line 320 is a weakened section of the cover 302 that bends easier than the remainder of the distal tip of the cover 302. For example, a trough can be formed in the circumferential interior 316 and/or the circumferential exterior 322 of the cover 302 to locally thin the cover 302. Thereby, the cover 302 elastically buckles at the fold line 320 (as shown in FIG. 11B) when the electrode 306 is in a deployed configuration and then returns to its original shape (as shown in FIG. 9) when the electrode 306 is in the stowed configuration.

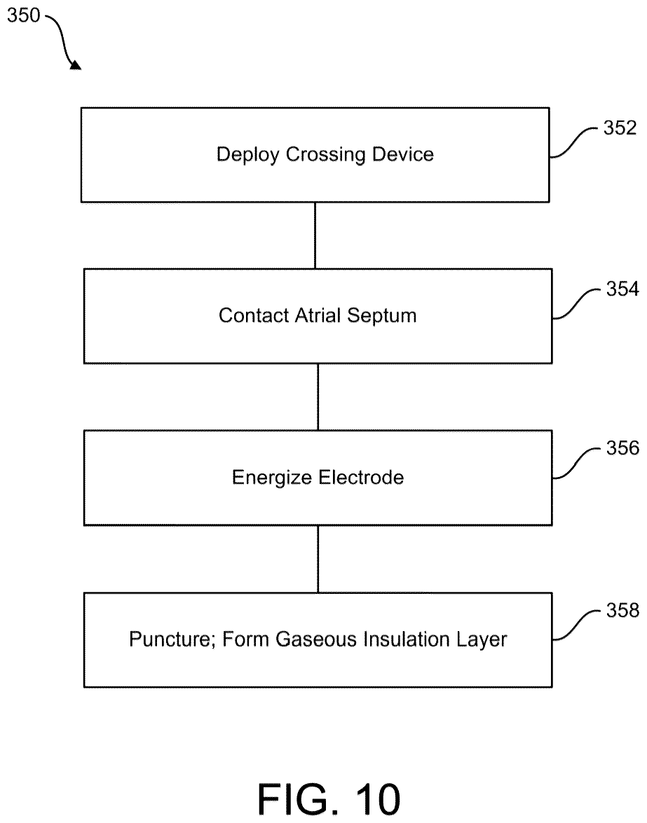

FIG. 10 shows a method 350 of crossing the atrial septum 136 using the crossing device 300. FIGS. 11A-11D show the operations of crossing the atrial septum 136. FIGS. 10 and 11A-11D will now be discussed in conjunction with one another, and each operation of the method 350 is illustrated by a corresponding one of FIGS. 11A-11D.

In the illustrated embodiment, the method 350 begins with operation 352 wherein the crossing device 300 is transformed to the deployed configuration. As shown in FIG. 11A, the physician has retracted the cover 302 (as indicated by the arrow) with respect to the central member 304 until the distal ends of the cover 302 and the central member 304 are coterminous.

At operation 354, the crossing device 300 is positioned in direct contact with the atrial septum 136. As shown in FIG. 11B, the physician has returned the proximal end of the cover 302 to its stowed location by moving the cover in the direction indicated by the arrow. As shown, the crossing device 300 is abutting the atrial septum 136, so the distal end of the cover 302 cannot return to its stowed location. Instead, the distal end of the cover 302 remains coterminous with the distal end surface 310 of the electrode 306. Thus, the cover 302 buckles and expands radially outward at the fold line 320.

In some embodiments, pressing the crossing device 300 against the atrial septum 136 results in the cover 302 moving from its stowed location into the configuration shown in FIG. 11B. In such embodiments, the operation 352 is not necessary to perform since it will occur automatically in the next operation (i.e., the corresponding equivalent of the operation 354).

At operation 356, as shown in FIG. 11C, the physician has activated the electrosurgical generator 104 (shown in FIG. 1) which energizes the electrode 306. Virtually all of the RF energy being transmitted to the electrode 306 flows into the cells of the atrial septum 136, because the electrode 306 is electrically insulated from the blood 270 by the cover 302. Concentrating the RF energy into the atrial septum 136 increases the efficiency and ease of the crossing. In particular, the crossing device 300 penetrates through the atrial septum 136 with minimal tenting of the atrial septum 136 and with minimal jumping of the crossing device 300 upon breakthrough. The ease of the puncture means that the physician does not need to exert as much force on the crossing device 300 compared to a prior art crossing system that has inefficient or diffuse RF energy delivery. The inefficiency or diffuseness of the prior art can result from insufficient insulation of the electrode from the blood 270. Such an electrode would maintain contact with the blood 270 even as it penetrates into the atrial septum 136, so only a fraction of the RF energy provided to the electrode would be delivered to the atrial septum 136. The inefficient or diffuse delivery of the RF energy can result in the tissue cells receiving an insufficient amount of power for vaporization but enough for the tissue cells to be desiccated instead. Desiccation increases the electrical resistance of the affected tissue cells, because there are fewer polar water molecules in the cells to conduct electricity, so the result is that even more power flows into the blood 270 instead of into the atrial septum 136.

In some embodiments, the time to puncture the atrial septum 136 can be about 300 ms to about 400 ms. Some electrosurgical generators, however, will continue to deliver power to the electrode 306 due to their programming and/or user selection/input. The total “on” time of such a system can be, for example, multiple seconds(s) (e.g., 2 s to 5 s), which means that the electrode 306 is still powered well after the puncture has been completed.

At operation 358, the puncture through the atrial septum 136 has been completed, and the cover 302 has returned to its original position. Thus, the electrode 306 is in the stowed configuration, but the open end of the cover 302 has allowed the blood 270 to contact the electrode 306. The RF energy being delivered now flows into the blood 270, which locally gasifies the blood 270. The vapor from the gasifying blood 270 form bubbles that collect and coalesce on the distal end surface 310 of the electrode 306 and the circumferential interior 316 of the cover 302. Once a sufficient amount of bubbles have coalesced, a gaseous insulation layer 370 is formed. The gaseous insulation layer 370 is positioned between the electrode 306 and the blood 270, so no more blood can enter the cover 302 and the electrode 306 is electrically insulated from the blood 270.

Until cessation of power delivery by the electrosurgical generator 104, voltage is still being applied to the electrode 306 at operation 358. But the current flow through the electrode 306 is greatly reduced compared to that of operation 356 since the electrode 306 is electrically insulated from the atrial septum 136 (by the cover 302) and the blood 270 (by the gaseous insulation layer 370). The great reduction in current flow means that less heat is produced, and less (if any) of the blood 270 is being locally gasified. Thus, the crossing device 300 essentially turns itself off once the puncture has been completed. The reduction in overall heat generation means that less heat is transferred to the cover 302, which allows for a material to be used for the cover 302 that is less heat resistant than in prior art crossing devices. Furthermore, once the crossing device 300 forms the gaseous insulation layer 370, the gaseous insulation layer 370 is retained in the cover 302. This retention is beneficial compared to prior art electrodes that are permanently coterminous with their electrical insulation, because such electrodes would be generating and releasing gaseous bubbles in the left atrium 138 of the heart 130 (shown in FIG. 2). Such free-floating bubbles can be dangerous to the patient 102 (shown in FIG. 1) for a variety of reasons.

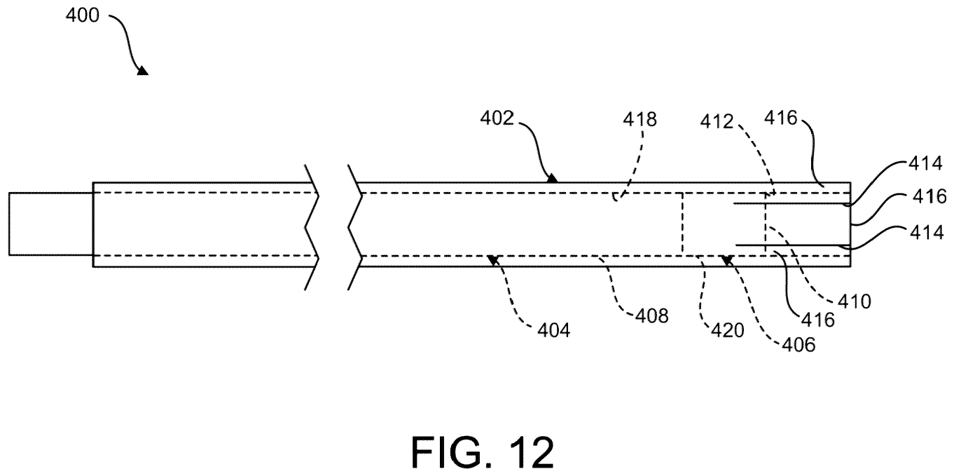

FIG. 12 shows an alternative crossing device 400 with a cover 402 and a central member 404 positioned within the cover 402 since the device 400 is in a stowed configuration. In the illustrated embodiment, the central member 404 comprises an electrode 406 at the distal end and a conductor 408 that is connected to the electrode 406. The electrode 406 is electrically connected to the electrosurgical generator 104 (shown in FIG. 1) via the conductor 408. Thereby, the central member 404 comprises, for example, stainless steel, nitinol, platinum, gold, iridium, or combinations thereof. In some embodiments, the conductor 408 includes its own electrical insulation, but in other embodiments, the conductor 408 is solely electrically insulated by the cover 402. In addition, the electrode 406 has a cylindrical shape with a flat distal end surface 410 having a distal circular edge 412, so the electrode 406 is atraumatic (i.e., not sharp). In other embodiments, however, the electrode has a distal domed shape.

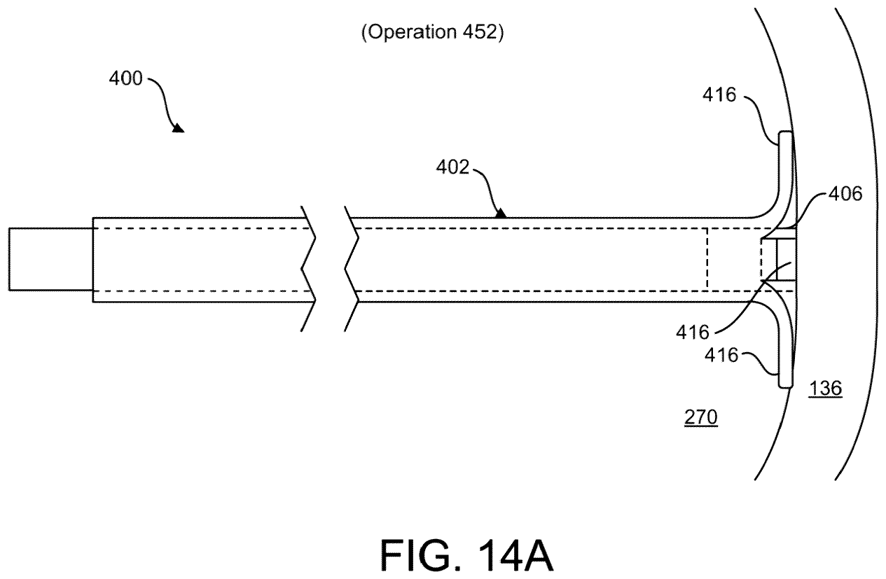

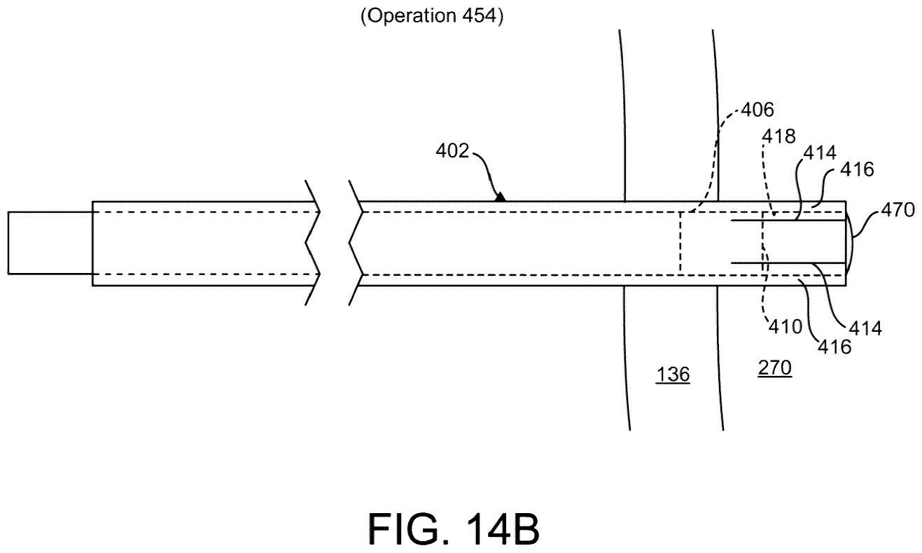

In the illustrated embodiment, the cover 402 is made from a compliant, electrically insulative material that extends distally beyond a distal end surface 410 of the electrode 406 when the electrode 406 is in the stowed configuration (as it is shown in FIG. 12). In some embodiments, the cover 402 extends at least one-quarter of a diameter (e.g., at least about 0.1 mm to 0.3 mm) beyond the distal end surface 410. The cover 402 includes a plurality of slits 414 that begin at the distal end of the cover 402 and extend proximally beyond the distal end surface 410 of the electrode 406 by at least half of a diameter (e.g., at least about 0.2 mm to 0.5 mm). The slits 414 are radially oriented and extend through the cover 402, so the distal end of the cover 402 is divided into a plurality of petals 416. While four, equally sized petals 416 are shown in FIG. 12, there can be fewer or greater petals in other embodiments. The petals 416 can be elastically splayed to deploy the electrode 406 (as shown in FIG. 14A), and then the petals 416 can return to their original shape (as shown in FIG. 12) to stow the electrode 406.

In the illustrated embodiment, there is little to no gap between the circumferential interior 418 of the cover 402 and the circumferential exterior 318 of the electrode 406 when the electrode 406 is in the stowed configuration. Thus, the circumferential exterior 420 is electrically insulated by the cover 402 when the electrode 406 is in the stowed configuration, because the conductive fluid medium cannot contact the circumferential exterior 420. The conductive fluid medium, however, can contact the distal end surface 410, because the distal end of the cover 402 is open.

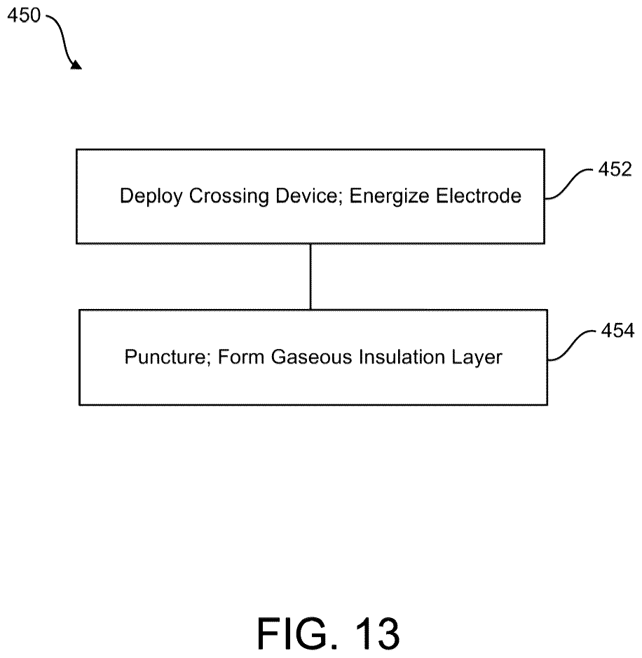

FIG. 13 shows a method 450 of crossing the atrial septum 136 using the crossing device 400. FIGS. 14A and 14B show the operations of crossing the atrial septum 136. FIGS. 13 and 14A and 14B will now be discussed in conjunction with one another, and each operation of the method 450 is illustrated by a corresponding one of FIGS. 14A and 14B.

In the illustrated embodiment, the method 450 begins with operation 452 wherein the crossing device 400 is transformed to the deployed configuration. As shown in FIG. 14A, the physician has positioned the crossing device 400 in contact with the atrial septum 136. In doing so, the petals 416 have splayed so that the electrode 406 is in direct contact with the atrial septum 136. In addition, the physician has activated the electrosurgical generator 104 (shown in FIG. 1) which energizes the electrode 406. A substantial portion of the RF energy being transmitted to the electrode 406 flows into the cells of the atrial septum 136 that are closest to the electrode 406, vaporizing the cells. At first, some of the RF energy is also transmitted into the blood 270 due to the gap between the cover 402 and the atrial septum 136. As the cells of the atrial septum 136 are vaporized at the distal end of the electrode 406, the crossing device 400 penetrates into the atrial septum 136.

The penetration into the atrial septum 136 closes the gap between the cover 402 and the atrial septum 136, so the electrode 406 is no longer in direct contact with the blood 270. Thereby, the electrode 406 is electrically insulated from the blood 270. Virtually all of the RF energy being transmitted to the electrode 406 flows into the cells of the atrial septum 136 which increases the efficiency and ease of the crossing. Even though the petals 416 will be folded back alongside a proximal portion of the cover 402, the crossing device 400 penetrates through the atrial septum 136 with minimal tenting of the atrial septum 136 and with minimal jumping of the crossing device 400 upon breakthrough. The ease of the puncture means that the physician does not need to exert as much force on the crossing device 400 compared to a prior art crossing system that has inefficient or diffuse RF energy delivery. The inefficiency or diffuseness of the prior art can result from insufficient insulation of the electrode from the blood 270. Such an electrode would maintain contact with the blood 270 even as it penetrates into the atrial septum 136, so only a fraction of the RF energy provided to the electrode would be delivered to the atrial septum 136. The inefficient or diffuse delivery of the RF energy can result in the tissue cells receiving an insufficient amount of power for vaporization but enough for the tissue cells to be desiccated instead. Desiccation increases the electrical resistance of the affected tissue cells, because there are fewer polar water molecules in the cells to conduct electricity, so the result is that even more power flows into the blood 270 instead of into the atrial septum 136.

In some embodiments, the time to puncture the atrial septum 136 can be about 300 ms to about 400 ms. Some electrosurgical generators, however, will continue to deliver power to the electrode 406 due to their programming and/or user selection/input. The total “on” time of such a system can be, for example, multiple seconds(s) (e.g., 2 s to 5 s), which means that the electrode 406 is still powered well after the puncture has been completed.

At operation 454, the puncture through the atrial septum 136 has been completed, and the cover 402 has returned to its original position wherein the petals 416 extend distally and contact each other along slits 414, respectively. Thus, the electrode 406 is in the stowed configuration, but the open end of the cover 402 has allowed the blood 270 to contact the electrode 406. The RF energy being delivered now flows into the blood 270, which locally gasifies the blood 270. The vapor from the gasifying blood 270 form bubbles that collect and coalesce on the distal end surface 410 of the electrode 406 and the circumferential interior 418 of the cover 402. Once a sufficient amount of bubbles have coalesced, a gaseous insulation layer 470 is formed. The gaseous insulation layer 470 is positioned between the electrode 406 and the blood 270, so no more blood can enter the cover 402 and the electrode 406 is electrically insulated from the blood 270.

Until cessation of power delivery by the electrosurgical generator 104, voltage is still being applied to the electrode 406 at operation 454. But the current flow through the electrode 406 is greatly reduced compared to that of operation 452 since the electrode 406 is electrically insulated from the atrial septum 136 (by the cover 402) and the blood 270 (by the gaseous insulation layer 470). The great reduction in current flow means that less heat is produced, and less (if any) of the blood 270 is being locally gasified. Thus, the crossing device 400 essentially turns itself off once the puncture has been completed. The reduction in overall heat generation means that less heat is transferred to the cover 402, which allows for a material to be used for the cover 402 that is less heat resistant than in prior art crossing devices. Furthermore, once the crossing device 400 forms the gaseous insulation layer 470, the gaseous insulation layer 470 is retained in the cover 402. This retention is beneficial compared to prior art electrodes that are permanently coterminous with their electrical insulation, because such electrodes would be generating and releasing gaseous bubbles in the left atrium 138 of the heart 130 (shown in FIG. 2). Such free-floating bubbles can be dangerous to the patient 102 (shown in FIG. 1) for a variety of reasons.

In an alternative embodiment to method 450, the order of the operations is different. For example, in some embodiments, the electrode 406 is energized in the stowed configuration, and then the electrode 406 is deployed into the atrial septum 136. In such an embodiment, there would be an additional gaseous insulation layer formed prior to contact with the atrial septum that would be destroyed during the puncturing.

It is well understood that methods that include one or more steps, the order listed is not a limitation of the claim unless there are explicit or implicit statements to the contrary in the specification or claim itself. It is also well settled that the illustrated methods are just some examples of many examples disclosed, and certain steps may be added or omitted without departing from the scope of this disclosure. Such steps may include incorporating devices, systems, or methods or components thereof as well as what is well understood, routine, and conventional in the art.

The connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C. The terms “couples,” “coupled,” “connected,” “attached,” and the like along with variations thereof are used to include both arrangements wherein two or more components are in direct physical contact and arrangements wherein the two or more components are not in direct contact with each other (e.g., the components are “coupled” via at least a third component), but still cooperate or interact with each other.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

We claim:

1. An electrosurgical system for puncturing tissue, the electrosurgical system comprising:
an electrosurgical generator configured to generate radiofrequency (RF) energy; and
a crossing device connected to the electrosurgical generator, the crossing device comprising:
an electrode positioned at a distal tip of the crossing device;
an electrically insulating cover that is elastically compliant, the cover extending distally beyond a distal end of the electrode when the electrode is in a stowed configuration, and the electrode extending distally beyond a distal end of the cover when the electrode is in a deployed configuration.

2. The electrosurgical system of claim 1, wherein the cover comprises a longitudinal tube that extends along a side of the electrode when the electrode is in a stowed position.

3. The electrosurgical system of claim 2, wherein:
the cover comprises a plurality of leaflets connected to the longitudinal tube and extending transversely over the distal end of the electrode when the electrode is in the stowed configuration; and
the plurality of leaflets is spread apart when the electrode is in the deployed configuration.

4. The electrosurgical system of claim 2, wherein the cover extends at least about one-quarter of a diameter of the electrode beyond the distal end of the electrode when the electrode is in the stowed configuration.

5. The electrosurgical system of claim 2, wherein the cover is corrugated when the electrode is in the deployed configuration.

6. The electrosurgical system of claim 2, wherein:
the distal end of the cover includes a plurality of slits defining a plurality of petals; and
the distal end of the cover is splayed when the electrode is in the deployed configuration.

7. The electrosurgical system of claim 1, wherein the cover is slidably positioned on the electrode.

8. The electrosurgical system of claim 1, wherein the crossing device is a wire.

9. The electrosurgical system of claim 1, wherein the crossing device is a needle.

10. A crossing device for an electrosurgical system for puncturing tissue, the crossing device comprising:
a conductor configured to be connected to an electrosurgical generator that is configured to generate radiofrequency (RF) energy;
an electrode electrically connected to the conductor and positioned at a distal end of the crossing device;
an electrically insulating cover that is elastically compliant and extends over the conductor and the electrode, the cover extending distally beyond a distal end of the electrode when the electrode is in a stowed configuration, and the electrode extending distally beyond a distal end of the cover when the electrode is in a deployed configuration.

11. The crossing device of claim 10, wherein the cover comprises a longitudinal tube that extends along a side of the electrode when the electrode is in a stowed position.

12. The crossing device of claim 11, wherein:
the cover comprises a plurality of leaflets connected to the longitudinal tube and extending transversely over the distal end of the electrode when the electrode is in the stowed configuration; and
the plurality of leaflets is spread apart when the electrode is in the deployed configuration.

13. The crossing device of claim 10, wherein the cover extends at least about one-quarter of a diameter of the electrode beyond the distal end of the electrode when the electrode is in the stowed configuration.

14. The electrosurgical system of claim 10, wherein the crossing device is a needle.

15. A method of operating an electrosurgical system for puncturing tissue, the method comprising:
deploying an electrode from an elastically compliant cover to expose an electrode;
energizing the electrode an electrosurgical generator; and
vaporizing tissue with the energized electrode.

16. The method of claim 15, wherein the electrode is positioned near to the tissue but not in contact with the tissue prior to energizing the electrode.

17. The method of claim 15, wherein the electrode is positioned in contact with the tissue prior to energizing the electrode.

18. The method of claim 15, wherein deploying the electrode comprises advancing the electrode and spreading apart a plurality of leaflets of the cover.

19. The method of claim 15, wherein deploying the electrode comprises retracting the cover.

20. The method of claim 15, wherein deploying the electrode comprises splaying a plurality of petals of the cover.
